SEQUENCE LISTING (12 sequences)

SEQ ID NO: 1 - RNA, 12 nt, synthetic construct
  source 1..12: mol_type = other RNA
ggcttctgacca

SEQ ID NO: 2 - RNA, 15 nt, synthetic construct
  source 1..15: mol_type = other RNA
ggccactcgttaaga

SEQ ID NO: 3 - RNA, 20 nt, synthetic construct
  source 1..20: mol_type = other RNA
ggccactgaagccattgaag

SEQ ID NO: 4 - RNA, 24 nt, synthetic construct
  source 1..24: mol_type = other RNA
ggccactgaagccattgtcaagga

SEQ ID NO: 5 - RNA, 25 nt, synthetic construct
  source 1..25: mol_type = other RNA
ggccactgaagccattgtcaccgaa

SEQ ID NO: 6 - RNA, 25 nt, synthetic construct
  source 1..25: mol_type = other RNA
ggcgaagcactcgtggccattcgca

SEQ ID NO: 7 - RNA, 10 nt, synthetic construct
  source 1..10: mol_type = other RNA
ggccaaggaa

SEQ ID NO: 8 - RNA, 11 nt, synthetic construct
  source 1..11: mol_type = other RNA
ggccaaggaaa

SEQ ID NO: 9 - RNA, 12 nt, synthetic construct
  source 1..12: mol_type = other RNA
ggccactgaaga

SEQ ID NO: 10 - RNA, 16 nt, synthetic construct
  source 1..16: mol_type = other RNA
ggccactgaagccatt

SEQ ID NO: 11 - RNA, 15 nt, synthetic construct
  source 1..15: mol_type = other RNA
ggccactgaaggaag

SEQ ID NO: 12 - RNA, 11 nt, synthetic construct
  source 1..11: mol_type = other RNA
cccgtaccccc

(19) United States
(12) Patent Application Publication
(10) Pub. No.: US 2025/0250614 A1
(43) Pub. Date: Aug. 7, 2025

(54) DETECTION OF MRNA PURITY IN A MIXTURE

(71) Applicant: c/o ModernaTX, Inc., Cambridge, MA (US)

(72) Inventor: Johnathan Goldman, Cambridge, MA (US)

(73) Assignee: c/o ModernaTX, Inc., Cambridge, MA (US)

(21) Appl. No.: 18/855,999

(22) PCT Filed: Apr. 10, 2023
(86) PCT No.: PCT/US2023/065593

§ 371 Date: Oct. 10, 2024

Related U.S. Application Data
(60) Provisional application No. 63/329,824, filed on Apr. 11, 2022.

(51) Int. Cl. C12Q 1/6818; C12N 15/10; C12N 15/11; C12Q 1/6876

(52) U.S. Cl. CPC C12Q 1/6818; C12N 15/101; C12N 15/11; C12Q 1/6876

(57) ABSTRACT

Provided herein are methods of detecting mRNA purity in a mixture and related constructs. Also provided are compositions for separating and detecting full length mRNA from a mixture.

### RELATED APPLICATION

This application claims the benefit under 35 U.S.C. § 119(e) to U.S. Provisional Application No. 63/329,824, entitled “DETECTION OF MRNA PURITY IN A MIXTURE,” filed on Apr. 11, 2022, the entire contents of which are incorporated herein by reference.

### REFERENCE TO AN ELECTRONIC SEQUENCE LISTING

The contents of the electronic sequence listing (M137870233WO00-SEQ-HCL.xml; Size: 11,545 bytes; and Date of Creation: Apr. 10, 2023) is herein incorporated by reference in its entirety.

### FIELD OF THE INVENTION

The invention involves the detection of mRNA based on a mobility-based separation of mRNA, methods of detecting mRNA purity in a mixture, and compositions of target mRNA molecules with tags and other tools useful in the methods.

### BACKGROUND

The purity of a target mRNA in a mixture may be determined by methods such as reverse-phase ion-pair high-performance liquid chromatography (RPIP-HPLC) and capillary electrophoresis (CE). These methods do not work well for complex products because the mRNAs in the mixture may co-elute in a complicated, size-based way. For example, degraded mRNAs close in size may not clearly separate and additionally mRNA may co-elute with impurities. Several obstacles remain to be overcome for the successful application of these methodologies for separating mRNA in a mixture.

### SUMMARY

A method for detecting a mRNA is provided in some aspects. The method comprises subjecting a liquid sample comprising the mRNA to mobility or charge based separation, wherein the mRNA comprises a first tag that increases hydrodynamic drag during the mobility or charge based separation and detecting the mRNA. In some embodiments the mobility or charge based separation is an electrophoresis method. In some embodiments the electrophoresis method is free solution electrophoresis.

In some embodiments, the method comprises subjecting a liquid sample comprising the mRNA to free solution electrophoresis, wherein the mRNA comprises a first tag that increases hydrodynamic drag during the electrophoresis and detecting the mRNA.

In some embodiments the first tag comprises a first nucleic acid sequence that is complementary to a first polynucleotide segment of the mRNA. In some embodiments the first polynucleotide segment comprises at least a first portion of a first untranslated region (UTR) of the mRNA. In some embodiments the first UTR is a 5′UTR. In some embodiments the first UTR is a 3′ UTR.

In some embodiments the first tag comprises a first hydrophobic region linked to the first nucleic acid sequence. In some embodiments the hydrophobic region comprises an alkyl group. In some embodiments the alkyl group is linear or branched. In some embodiments the alkyl group is saturated. In some embodiments the alkyl group comprises 8-24 carbon atoms, 12-24 carbon atoms, 15-24 carbon atoms, 18-24 carbon atoms, 8-18 carbon atoms, 12-18 carbon atoms, 15-18 carbon atoms, 8-10 carbon atoms, 8-12 carbon atoms, or 12-18 carbon atoms. In some embodiments the hydrophobic region comprises a C18 molecule. In some embodiments the hydrophobic region comprises two C18 molecules.

In some embodiments the first nucleic acid sequence comprises a DNA oligonucleotide, optionally comprising a DNA base modification, LNA base modification, or 2′ Ome base modification.

In some embodiments the mRNA comprises a second tag. In some embodiments the second tag comprises a second nucleic acid sequence that is complementary to a second polynucleotide segment of the mRNA. In some embodiments the second polynucleotide segment comprises at least a second portion of a second untranslated region (UTR) of the mRNA comprises, wherein optionally the first UTR and second UTR are the same or different UTRs. In some embodiments the second UTR is a 5′UTR. In some embodiments the second UTR is a 3′ UTR. In some embodiments the second tag comprises a detectable tag attached to the second nucleic acid sequence, optionally wherein the tag is a non-fluorescent tag.

In some embodiments the first or second tag comprises a tag which absorbs at a specific UV wavelength attached to the nucleic acid sequence, wherein the wavelength is distinct from a RNA wavelength. In some embodiments the first or second tag comprises a spectral tag attached to the nucleic acid sequence. In some embodiments the fluorescent tag is an RNA specific fluorescent tag. In some embodiments the fluorescent tag is a 6-Carboxyfluorescein (6-FAM) fluorescent tag. In some embodiments the fluorescent tag is selected from 5-TAMRA, 6-F.AM, Cy3, Cy5, Fluorescein, TYE 563, TYE 664, TYE 705, and Yakima Yellow. In some embodiments second tag comprises an RNA dye. In some embodiments the second tag comprises a hydrophobic region linked to the nucleic acid sequence. In some embodiments the hydrophobic region comprises an alkyl group.

In some embodiments the electrophoresis is a capillary electrophoresis assay. In some embodiments the capillary electrophoresis assay is an end labeled free solution electrophoresis (ELFSE) assay. In some embodiments the capillary electrophoresis assay is a micellar end labeled free solution electrophoresis (miELFSE) assay.

In some embodiments the mRNA is a full-length mRNA. In some embodiments the full-length mRNA is tagged with the first and the second tag. In some embodiments the first polynucleotide segment comprises at least a portion of a 5′UTR of the mRNA and the second polynucleotide segment comprises at least a portion of a 3′UTR of the mRNA. In some embodiments the first polynucleotide segment comprises at least a portion of a 3′UTR of the mRNA and the second polynucleotide segment comprises at least a portion of a 5′UTR of the mRNA. In some embodiments detecting the mRNA comprises detecting separation properties of the mRNA based on mobility and detecting a spectral signal.

In some embodiments detecting the target mRNA comprises detecting separation properties of the target mRNA, based on the mobility of the target mRNA. In some embodiments the liquid sample comprises 1-20, 1-15, 1-10, 1-5, 5-20, 5-15, 5-10, 10-20, 10-15, or 15-20 mRNAs. In some embodiments the liquid sample comprises 1, 2, 3, 4, 5, 6, 7, 8, 9, 10, 11, 12, 13, 14, 15, 16, 17, 18, 19, 20 mRNAs. In some embodiments the liquid sample comprises 1-20, 1-15, 1-10, 1-5, 5-20, 5-15, 5-10, 10-20, 10-15, or 15-20 mRNAs, and wherein at least two of the mRNAs comprise a first tag, wherein each of the first tags is distinct from one another. In some embodiments the liquid sample comprises 1, 2, 3, 4, 5, 6, 7, 8, 9, 10, 11, 12, 13, 14, 15, 16, 17, 18, 19, or 20 mRNAs, and wherein at least two of the mRNAs comprise a first tag, wherein each of the first tags is distinct from one another. In some embodiments the liquid sample comprises 1-20 mRNAs, and wherein at least two of the mRNAs comprise a first tag, wherein each of the first tags is distinct from one another.

In some embodiments the first tag and/or the second tag are attached to the sample mRNA using an annealing procedure, wherein the annealing procedure may comprise incubating the sample mRNA with the first tag and/or the second tag under hybridization conditions.

In some embodiments, the second tag comprises a nucleic acid sequence that is complementary to a polynucleotide segment of the target mRNA in the 3′ UTR.

In some embodiments, the first tag and the second tag are attached to the sample mRNA using an annealing procedure. In some embodiments, the annealing procedure comprises incubating the sample mRNA with the first tag and the second tag at 75° C. with 25 mM KCl in TE. In some embodiments, the tagged mRNA is introduced to the buffer solution by an injection method. In some embodiments, the injection method is a pressure injection. In some embodiments, the injection method is an electrokinetic injection.

In some embodiments, the buffer solution comprises a plurality of reagents comprising a first surfactant and a second surfactant. In some embodiments, the buffer solution is a CiEJ buffer solution optionally containing urea. In some embodiments, the first surfactant is pentaethylene glycol monododecyl ether. In some embodiments, the second surfactant is pentaethylene glycol monodecyl ether. In some embodiments, the first surfactant is pentaethylene glycol monodecyl ether. In some embodiments, the second surfactant is pentaethylene glycol monododecyl ether.

In some embodiments, the buffer solution interacts with the hydrophobic region to form a drag tag. In some embodiments, the drag tag is a micelle drag tag. In some embodiments, the drag tag is any large molecule. In some embodiments, the drag tag is any large, uncharged molecule. In some embodiments the drag tag is protein(s), polymeric nanoparticles, and metal nanoparticles. In some embodiments the drag tag is protein(s). In some embodiments the drag tag is polymeric nanoparticles. In some embodiments the drag tag is metal nanoparticles. In some embodiments, the drag tag is selected from a worm-like micelle, a spherical micelle or a lamellar micelle. In some embodiments, the drag tag is a cylindrical worm-like micelle. In some embodiments, the signal corresponding to the target mRNA is based on a mobility-base separation.

In some embodiments, the target mRNA is greater than 500 nucleotides. In some embodiments, the mRNA is at least 500 nucleotides. In some embodiments the mRNA is about 500-15000 (500-1500, 500-12000, 500-10000, 500-8000, 500-5000, 500-1000, 1000-15000, 1000-12000, 1000-10000, 1000-8000, 8000-15000, 8000-12000, 8000-10000, 1000-15000, 10000-12000, 12000-15000) nucleotides. In some embodiments, the target mRNA is 500-15,000 nucleotides, 500-12,000 nucleotides, 500-10,000 nucleotides, 500-8,000 nucleotides, 1,000-15,000 nucleotides, 1,000-12,000 nucleotides, 1,000-10,000 nucleotides or 1,000-8,000 nucleotides. In some embodiments, the mRNA is about 500, 1000, 8000, 10000, 12000, or 15000 nucleotides. In some embodiments, the mRNA is about 500 nucleotides. In some embodiments, the mRNA is about 1000 nucleotides. In some embodiments, the mRNA is about 8000 nucleotides. In some embodiments, the mRNA is about 10000 nucleotides. In some embodiments, the mRNA is about 12000 nucleotides. In some embodiments, the mRNA is about 15000 nucleotides.

Provided herein, in some aspects is a construct comprising, a mRNA polynucleotide, a hydrophobic tag, and a detectable tag. In some embodiments, the mRNA polynucleotide is a full-length mRNA polynucleotide and comprises a 5′ UTR, a polynucleotide sequence, a 3′ UTR and a poly-A tail.

Provided herein, in some aspects, are methods for detecting a mRNA in a mixture, comprising: (a) attaching a first tag and a second tag to a sample mRNA molecule to generate a tagged mRNA molecule, (b) subjecting the tagged mRNA molecule to a capillary electrophoresis assay, wherein the first tag causes a change in separation properties of the mRNA molecule in the assay to separate the mRNA molecule from other components of the mixture, and (c) detecting a signal corresponding to the second tag based on the separated mRNA molecule, and thereby identifying a signal corresponding to the target mRNA.

Provided herein, in some aspects are compositions, comprising a nucleic acid sequence that is complementary to a polynucleotide segment of a target mRNA and a hydrophobic region linked to the nucleic acid sequence, wherein the hydrophobic region comprises an alkyl group.

In some embodiments, the polynucleotide segment comprises at least a portion of an untranslated region (UTR) of the mRNA. In some embodiments, the UTR is a 5′UTR. In some embodiments, the UTR is a 3′ UTR.

In some embodiments, the alkyl group is linear. In some embodiments, the alkyl group is saturated. In some embodiments, the alkyl group comprises 8-24 carbon atoms, 12-24 carbon atoms, 15-24 carbon atoms, 18-24 carbon atoms, 8-18 carbon atoms, 12-18 carbon atoms, 15-18 carbon atoms, 8-10 carbon atoms, 8-12 carbon atoms, or 12-18 carbon atoms. In some embodiments, the alkyl group is branched. In some embodiments, the hydrophobic region comprises a C18 molecule, two C18 molecules or more than two C18 molecules. In some embodiments, the hydrophobic region comprises a DNA base modification, LNA base modification or 2′ Ome base modification.

In some embodiments the nucleic acids comprise base modifications to alter the melting temperature of the duplex formed by hybridization. In some embodiments the base modification comprises a hydrophobic moiety or a fluorescent moiety.

In some aspect a construct comprising: (i) a mRNA polynucleotide, (ii) a first tag comprising a hydrophobic region linked to a first nucleic acid sequence hybridized to the mRNA polynucleotide, wherein the first nucleic acid sequence is complementary to a first polynucleotide segment of the mRNA and (iii) a second tag comprising detectable molecule linked to a second nucleic acid sequence hybridized to the mRNA polynucleotide, wherein the second nucleic acid sequence is complementary to a second polynucleotide segment of the mRNA is provided.

Each of the limitations of the invention can encompass various embodiments of the disclosure. It is, therefore, anticipated that each of the limitations of the disclosure involving any one element or combinations of elements can be included in each aspect or embodiment of the invention. This disclosure is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

FIG. 1 is an electropherogram of mRNA-1 tagged with 6-FAM detectable tag and C18 hydrophobic tag.

FIG. 2A is an electropherogram of mRNA-2 tagged with a sequence specific C18 hydrophobic tag.

FIG. 2B is an electropherogram of mRNA-3 subject to the annealing procedure in the presence of with a mRNA-2 sequence specific C18 hydrophobic tag.

FIG. 3 is a schematic of a tagged mRNA, wherein one tag is attached to the 5′ end and a second tag is attached to the 3′ end of the mRNA.

### DETAILED DESCRIPTION

The present disclosure includes methods and compositions for detecting a mRNA in a mixture, for instance in order to determine sample purity. In some embodiments, the mRNA being detected is a target mRNA. For example, separating full-length mRNA from complex mixtures, including for instance truncated RNAs can be challenging. The use of size-based separation techniques may be inadequate especially when RNAs in a mixture have similar sizes. Methods for adequately separating mRNAs to determine purity of an mRNA product are disclosed herein.

Aspects of the present disclosure relate to methods of detecting target mRNAs in a sample using separation methods, such as methods which achieve separation of molecules based on mobility and/or charge. In some embodiments, the target mRNA are non-covalently tagged in order to produce a drag tag during the separation process, which enables separation of different nucleic acids. For example, RNA specifically moves in a free solution electrophoresis method according to charge, and surface area/mass ratio. The formation of a drag tag on the tagged target mRNA results in the production of a greater mobility/charge differential between a target mRNA and other nucleic acids in the mixture or sample. As a result, the presence of the target mRNA in the sample using this separation method can be accurately determined without interference of other nucleic acids. For instance, a target mRNA can be separated from other mRNAs as well as fragments of the target mRNA and any other nucleic acid using this method. The methods may also be used to determine whether an mRNA is a full-length version, rather than a fragment.

In some embodiments, the method involves the non-covalent attachment of a set of tags to a target mRNA in a mixture. The tags may be comprised of a nucleic acid which is complementary to an RNA, or a portion thereof, in order to achieve the non-covalent attachment. One or more of the tags also has a hydrophobic tail, which can interact with components of a buffer solution to form a drag tag, such as a micelle structure. The drag tag allows for the separation of the mRNA from where it would normally elute in free solution. Such methods have not previously been achieved on large nucleic acids, for instance greater than 500 bp and/or on mRNA.

Tagged mRNA

Aspects of the disclosure involve subjecting a mixture such as a liquid sample comprising a tagged mRNA to electrophoresis, such that the tagged mRNA increases the hydrodynamic drag during the electrophoresis to enable the detection of the target mRNA. The target mRNA is labeled with one or more tags. In some embodiments, the tagged mRNA comprises a first tag that increases hydrodynamic drag during the free solution electrophoresis. In other embodiments, the tagged mRNA comprises a second tag. In some embodiments, the tagged mRNA comprises a first tag and a second tag. In some embodiments, a full-length mRNA is tagged with a first tag. In some embodiments, a full-length mRNA is tagged with a second tag. In some embodiments, a full-length mRNA is tagged with a first tag and a second tag. A tag as used herein comprises an mRNA-specific region linked to a detectable component. A detectable component as used herein refers to a component that is capable of being detected by any means, including but not limited to fluorescence detection and detection by shift in migration time. An mRNA-specific region is a portion of the tag that recognizes and binds to the target mRNA in a specific manner. The mRNA-specific region is designed to identify a specific portion of the target mRNA sequence. In some embodiments, the mRNA specific region is a nucleic acid. In some embodiments, the tag comprises a nucleic acid sequence that is complementary to a polynucleotide segment of the target mRNA. In some embodiments, the first tag comprises a nucleic acid sequence that is complementary to a first polynucleotide segment of the target mRNA. In some embodiments, the second tag comprises a second nucleic acid sequence that is complementary to a second polynucleotide segment of the mRNA. The term “complementary” as used herein refers to the hybridization or base pairing between nucleotides or nucleic acids, such as between the two strands of a double stranded DNA molecule or between a DNA oligonucleotide and a portion of a target, such as a target mRNA sequence. Complementary nucleotides are, generally, A and T (or A and U), or C and G. In some embodiments, at least one non-covalent bond formed between the mRNAs of an oligonucleotide-mRNA hybrid is a result of Watson-Crick base-pairing. The term “Watson-Crick base-pairing”, or “base-pairing” refers to the formation of hydrogen bonds between specific pairs of nucleotide bases (“complementary base pairs”). For example, two hydrogen bonds form between adenine (A) and uracil (U), and three hydrogen bonds form between guanine (G) and cytosine (C). One method of assessing the strength of bonding between two polynucleotides is by quantifying the percentage of bonds formed between the guanine and cytosine bases of the two polynucleotides (“GC content”). In some embodiments, the GC content of bonding between the two nucleic acids is at least 10%, at least 20%, at least 30%, at least 40%, or at least 50%.

Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90%, 91%, 92%, 93%, 94%, 95%, 96%, 97%, 98% and more preferably about 99% or 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% 91%, 92%, 93%, 94%, 95%, 96%, 97%, 98%, 99% or 100% complementary.

The “percent identity,” “sequence identity,” “% identity,” or “% sequence identity” (as they may be interchangeably used herein) of two sequences (e.g., nucleic acid or amino acid) refers to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range.

The term “hybridization” or “annealing” as used herein refers to the process in which two single-stranded polynucleotides bind non-covalently to form a stable double-stranded polynucleotide. Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25° C. to 30° C. are suitable for allele-specific probe hybridizations or conditions of 100 mM MES, 1 M Na⁺, 20 mM EDTA, 0.01% Tween-20 and a temperature of 30° C. to 50° C., preferably at about 45° C. to 50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. In some embodiments, the tags may be attached to the target mRNA in an annealing or hybridization procedure. In some embodiments, the tags may be attached to the target mRNA in an annealing procedure. In some embodiments, the tags may be attached to the target mRNA in a hybridization procedure. In some embodiments, the first tag and/or the second tag are attached to the sample mRNA using a hybridization procedure. In some embodiments, the first tag is attached to the sample mRNA using a hybridization procedure. In some embodiments, the second tag is attached to the sample mRNA using a hybridization procedure. In some embodiments, the first tag and/or the second tag are attached to the sample mRNA using an annealing procedure. In some embodiments, the first tag is attached to the sample mRNA using an annealing procedure. In some embodiments, the second tag is attached to the sample mRNA using an annealing procedure. In some embodiments, the annealing procedure comprises incubating the sample mRNA with the first tag and/or the second tag under hybridization conditions. In some embodiments, the annealing procedure comprises incubating the sample mRNA with the first tag under hybridization conditions. In some embodiments, the annealing procedure comprises incubating the sample mRNA with the second tag under hybridization conditions.

The annealing procedure may include incubating the mRNA with one or more tags at, for instance, 75° C. with 25 mM KCl in TE. In some embodiments, the annealing procedure includes incubating the mRNA with one or more tags at, at a temperature (e.g., 75° C.) with a concentration of KCl (e.g., 25 mM) in a buffer (e.g., TE). In some embodiments, the temperature is about 73-77, 73-76, 73-75, 73-74, 74-77, 74-76, 74-75, 75-77, 75-76, or 76-77° C. In some embodiments, the temperature is about 73, 74, 75, 76, or 77° C. In some embodiments, the temperature is about 73° C. In some embodiments, the temperature is about 74° C. In some embodiments, the temperature is about 75° C. In some embodiments, the temperature is about 76° C. In some embodiments, the temperature is about 77° C. In some embodiments, the concentration of KCl is about 25, 20-35, 20-30, 20-25, 25-35, 25-30, 30-35 or mM. In some embodiments, the concentration of KCl is about 20, 25, 30, 35 mM. In some embodiments, the concentration of KCl is about 20 mM. In some embodiments, the concentration of KCl is about 25 mM. In some embodiments, the concentration of KCl is about 30 mM. In some embodiments, the concentration of KCl is about 35 mM. In some embodiments, the hybridization conditions comprise a temperature of 73-77° C., about 25 mM KCl in a buffer.

The nucleic acid sequence of the tag is, in some embodiments, a deoxyribonucleic acid (DNA), ribonucleic acid (RNA), or hybrid DNA-RNA oligonucleotide. An “oligonucleotide” is a single stranded nucleic acid (sometimes referred to as “polynucleotide” or “nucleic acid sequence”) ranging from at least 2 to about 100 nucleotides in length. In some embodiments, the oligonucleotide is 3-100, 4-100, 5-100, 6-100, 7-100, 8-100, 9-100, 10-100, 20-100, 30-100, 40-100, 50-100, 60-100, 70-100, 80-100, 90-100, 3-50, 4-50, 5-50, 6-50, 7-50, 8-50, 9-50, 10-50, 20-50, 30-50, 40-50, 3-40, 4-40, 5-40, 6-40, 7-40, 8-40, 9-40, 10-40, 20-40, 30-40, 3-30, 4-30, 5-30, 6-30, 7-30, 8-30, 9-30, 10-30, 20-30, 25-30, 3-20, 4-20, 5-20, 6-20, 7-20, 8-20, 9-20, 10-20, or 15-20. The oligonucleotides can include sequences isolated from natural sources, recombinantly produced, or artificially synthesized, and mimetics thereof.

In some embodiments, the oligonucleotide is a messenger RNA (mRNA). In some embodiments, the mRNA is at least 500 nucleotides. In some embodiments the mRNA is about 500-15000, 500-1500, 500-12000, 500-10000, 500-8000, 500-5000, 500-1000, 1000-15000, 1000-12000, 1000-10000, 1000-8000, 8000-15000, 8000-12000, 8000-10000, 1000-15000, 10000-12000, or 12000-15000) nucleotides. In some embodiments, the mRNA is about 500, 1000, 8000, 10000, 12000, or 15000 nucleotides. In some embodiments, the mRNA is about 500 nucleotides. In some embodiments, the mRNA is about 1000 nucleotides. In some embodiments, the mRNA is about 8000 nucleotides. In some embodiments, the mRNA is about 10000 nucleotides. In some embodiments, the mRNA is about 12000 nucleotides. In some embodiments, the mRNA is about 15000 nucleotides.

The oligonucleotides may be composed of naturally occurring bases, or optionally may comprise a DNA base modification, LNA base modification, PNA (peptide nucleic acid) modification, or 2′ Ome base modification. In some embodiments, the nucleic acid sequence comprises a DNA oligonucleotide. In some embodiments, the nucleic acid sequence comprises a DNA base modification, LNA base modification, or 2′ Ome base modification. In some embodiments, the nucleic acid sequence comprises a DNA base modification. In some embodiments, the nucleic acid sequence comprises a LNA base modification. In some embodiments, the nucleic acid sequence comprises a 2′ Ome base modification.

In some embodiments, the first nucleic acid sequence comprises a DNA oligonucleotide. In some embodiments, the first nucleic acid sequence comprises a DNA base modification, LNA base modification, or 2′ Ome base modification. In some embodiments, the first nucleic acid sequence comprises a DNA base modification. In some embodiments, the first nucleic acid sequence comprises a LNA base modification. In some embodiments, the first nucleic acid sequence comprises a 2′ Ome base modification.

In some embodiments, the second nucleic acid sequence comprises a DNA oligonucleotide. In some embodiments, the second nucleic acid sequence comprises a DNA base modification, LNA base modification, or 2′ Ome base modification. In some embodiments, the second nucleic acid sequence comprises a DNA base modification. In some embodiments, the second nucleic acid sequence comprises a LNA base modification. In some embodiments, the second nucleic acid sequence comprises a 2′ Ome base modification.

The nucleic acid sequence of the tag may be complementary to any portion of the target mRNA. In some embodiments, the nucleic acid sequence is complementary to at least a portion of an untranslated region (UTR) of the target mRNA. UTRs are sections of a nucleic acid before a start codon (5′ UTR) and after a stop codon (3′ UTR) that are not translated. In some embodiments, a target mRNA of the disclosure comprises an open reading frame (ORF) encoding one or more proteins or peptides further comprises one or more UTR (e.g., a 5′ UTR or functional fragment thereof, a 3′ UTR or functional fragment thereof, or a combination thereof), and the tag is complementary to a UTR sequence.

The terms 5′ and 3′ are used herein to describe features of a nucleic acid sequence related to either the position of genetic elements, such as e.g., 5′ UTR or 3′ UTR, and/or the direction of events (5′ to 3′), such as transcription by RNA polymerase or translation by the ribosome which proceeds in 5′ to 3′ direction. Synonyms are upstream (5′) and downstream (3′). Conventionally, DNA sequences, gene maps, vector cards, and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

In some embodiments, the nucleic acid sequence in the tag may anneal to the target mRNA in the 5′ UTR or 3′ UTR. In some embodiments, the nucleic acid sequence may anneal to the target mRNA at the 5′ end of the 5′ UTR or the 3′ end of the 3′ UTR. When a nucleic acid sequence is annealed at the 5′ end of the 5′UTR, the nucleotide at the 3′ end of the nucleic acid sequence base pairs with the nucleotide at the 5′ end of the 5′UTR. When a nucleic acid sequence is annealed at the 3′ end of the 3′UTR, the nucleotide at the 5′ end of the nucleic acid sequence base pairs with the nucleotide at the 3′ end of the 3′UTR. An advantage of using a nucleic acid sequence that anneals to the target mRNA at the 5′ end of the 5′ UTR is that it probes the fidelity of the mRNA at the 5′ end. Using standard methods for detecting mRNA, when the 5′ end of an mRNA is missing or has an error, it may not be possible to distinguish the defective mRNA from the pure full-length mRNA because it will co-elute with the full-length. Using the methods of the disclosure with the tag that binds to the 5′UTR, however, the mRNA having a defective 5′ end will be detected.

When two tags are used in which one binds to the 5′ end of the 5′ UTR and the other binds to the 3′ end of the 3′ UTR, the methods can be used to accurately detect full-length mRNA. Truncated mRNA or fragments of mRNA that have errors or missing sequences at the 3′ and/or 5′ end will not be detected. Thus, the mRNA detected using the methods disclosed herein is more pure than using existing methods.

In some embodiments, the first polynucleotide segment comprises at least a first portion of a first untranslated region (UTR) of the mRNA. In some embodiments, first UTR is a 5′ UTR. In some embodiments, the first UTR is a 3′ UTR. In some embodiments, the second polynucleotide segment comprises at least a second portion of a second untranslated region (UTR) of the mRNA comprises, wherein the first UTR and second UTR are the same or different UTRs. In some embodiments, the first UTR and second UTR are the same UTRs. In some embodiments, the first UTR and second UTR are different UTRs. In some embodiments, the first UTR and second UTR are both 5′ UTRs. In some embodiments, the first UTR and second UTR are both 3′ UTRs. In some embodiments, the first UTR and second UTR are different UTRs. In some embodiments, the first UTR is a 5′ UTR and second UTR is a 3′ UTR. In some embodiments, the first UTR is a 3′ UTR and second UTR is a 5′ UTR. In some embodiments, the second UTR is a 5′ UTR. In some embodiments, the second UTR is a 3′ UTR. In some embodiments, the polynucleotide segment comprises at least a portion of an untranslated region (UTR) of the mRNA. In some embodiments, the UTR is a 5′ UTR. In some embodiments, the UTR is a 3′ UTR.

In some embodiments, the first polynucleotide segment comprises at least a portion of a 5′UTR of the mRNA. In some embodiments, the second polynucleotide segment comprises at least a portion of a 3′UTR of the mRNA. In some embodiments, the first polynucleotide segment comprises at least a portion of a 3′UTR of the mRNA. In some embodiments, the second polynucleotide segment comprises at least a portion of a 5′UTR of the mRNA. In some embodiments, the first polynucleotide segment comprises at least a portion of a 5′UTR of the mRNA and the second polynucleotide segment comprises at least a portion of a 3′UTR of the mRNA. In some embodiments, the first polynucleotide segment comprises at least a portion of a 3′UTR of the mRNA and the second polynucleotide segment comprises at least a portion of a 5′UTR of the mRNA.

In some embodiments, the nucleic acid sequence of the tag may be complementary to an internal sequence of the target mRNA. It may be useful to probe an internal sequence, i.e., a sequence in the ORF when trying to detect presence of a target mRNA in a mixture of multiple types of mRNA. Several mRNA's in a mixed sample can be tagged and distinguished from one another.

The detectable component of the tag, in some embodiments, is a hydrophobic region or a label such as a fluorescent molecule. In some embodiments, the hydrophobic region is linked to the nucleic acid sequence. In some embodiments, the first tag comprises a hydrophobic region linked to the nucleic acid sequence. In some embodiments, the second tag comprises a hydrophobic region linked to the nucleic acid sequence. In some embodiments, the first tag comprises a first hydrophobic region linked to the nucleic acid sequence. In some embodiments, the second tag comprises a second hydrophobic region linked to the nucleic acid sequence. As described herein, a “hydrophobic region” refers to a discrete component of the tag, having hydrophobic properties. In some embodiments, the tag containing a hydrophobic region may be referred to as a “hydrophobic tag”. A hydrophobic molecule typically has a non-polar surface that repels water. Because the hydrophobic molecules cannot form hydrogen bonds with water the hydrophobic molecules in solution lump together. Molecules such as lipids, having hydrophobic and hydrophilic portions in a solution can be used to create a barrier between the hydrophobic molecule and water molecules, forming a structure with hydrophilic portions on the external surface.

In some non-limiting embodiments, the hydrophobic moiety comprises one or more alkyl groups. In some embodiments, the hydrophobic region comprises an alkyl group. In some embodiments, the first hydrophobic region comprises and alkyl group. In some embodiments, the second hydrophobic region comprises an alkyl group. The alkyl group can be linear and/or saturated. In some embodiments, the alkyl group is linear. In some embodiments, the alkyl group is branched. In some embodiments, the alkyl group is saturated. In some embodiments, the first hydrophobic region comprises an alkyl group. In some embodiments, the first hydrophobic region comprises a linear or branched alkyl group. In some embodiments, the first hydrophobic region comprises a linear alkyl group. In some embodiments, the first hydrophobic region comprises a branched alkyl group. In some embodiments, the first hydrophobic region comprises a saturated alkyl group. In some embodiments, the hydrophobic group is an alkyl group of at least about 8, 9, 10, 11, 12, 13, 14, 15, 16, 17, 18, 19, 20, 21, 22, 23, 24, 25, 26, 27, 28, 29, 30, 31, 32, 33, 34, 35 36, 37, 38, 39 or 40 carbon atoms or any range of any of the foregoing integers. In some embodiments, the hydrophobic group is an alkyl group of about 8-40, 8-30, 8-24, 8-20, 8-18, 8-12, 8-10, 10-40, 10-30, 10-24, 10-20, 10-12, 12-40, 12-18, 12-30, 12-24, 12-20, 15-18, 15-24, 18-24, or 20-24 carbon atoms. In some embodiments, the alkyl group comprises about 8-24 carbon atoms. In some embodiments, the alkyl group comprises about 12-24 carbon atoms. In some embodiments, the alkyl group comprises about 15-24 carbon atoms. In some embodiments, the alkyl group comprises about 18-24 carbon atoms. In some embodiments, the alkyl group comprises about 8-18 carbon atoms. In some embodiments, the alkyl group comprises about 12-18 carbon atoms. In some embodiments, the alkyl group comprises about 15-18 carbon atoms. In some embodiments, the alkyl group comprises about 8-10 carbon atoms. In some embodiments, the alkyl group comprises about 8-12 carbon atoms. In some embodiments, the alkyl group comprises about 8 carbon atoms.

For example, the alkyl group can be selected from the group consisting of an octyl, a nonyl, a decyl, an undecyl, a dodecyl, a tridecyl, a tetradecyl, a pentadecyl, a hexadecyl, a heptadecyl, an octadecyl, a nonadecyl, an icosyl, a henicosyl, a docosyl, a tricosyl and a tetracosyl group. In some embodiments, the tag has two alkyl groups, which may be the same or different, such as, for instance, two C18 groups. In some embodiments, the tag has more than two alkyl groups, which may be the same or different. In some embodiments, the hydrophobic region comprises a C18 molecule. In some embodiments, the hydrophobic region comprises one C18 molecule. In some embodiments, the hydrophobic region comprises two C18 molecules. In some embodiments, the hydrophobic region comprises more than two C18 molecules. In some embodiments, the hydrophobic region comprises about 1, 2, 3, 4, 5, 6, 7, 8, 9, 10, 11, or 12 C18 molecules. In some embodiments, the first hydrophobic region comprises a C18 molecule. In some embodiments, the first hydrophobic region comprises one C18 molecule. In some embodiments, the first hydrophobic region comprises two C18 molecules. For instance, the tag may comprise:

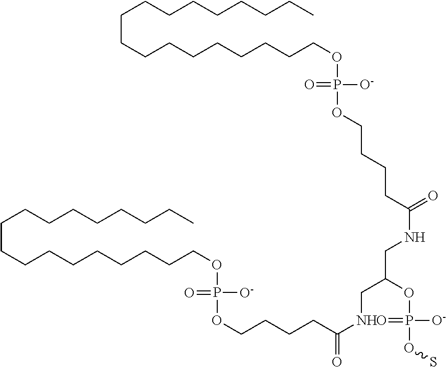 [SMILES: CCCCCCCCCCCCCCCCCCOP(=O)([O-])OCCCCC(=O)NCC(CNC(=O)CCCCOP(=O)([O-])OCCCCCCCCCCCCCCCCCC)OP(=O)([O-])OS]

In some embodiments, the hydrophobic group may comprise a functional group. In some non-limiting embodiments, the functional group is a chromophore. In other non-limiting embodiments, the functional group is a fluorophore. In other non-limiting embodiments, the functional group is boron-dipyrromethene. For example, the hydrophobic moiety can comprise a Bodipy fluorophore. In other non-limiting embodiments, the functional group can comprise a radioactive atom, such a ³²P or ³³P.

The hydrophobic group may be linked to a sugar of the nucleic acid. In some embodiments, the hydrophobic group is linked to the 3′ end of the nucleic acid. In some embodiments, the hydrophobic group is linked to the 5′ end of the nucleic acid. In some embodiments, a plurality of hydrophobic groups are linked to the nucleic acid. The linkage may be direct or may be through a linker. A linker is a group that bonds the hydrophobic group and the nucleoside. Non-limiting examples of linker groups include an amide, a phosphoramidite bond, and a phosphodiester bond.

In some embodiments, a second tag is used. The second tag can comprise a nucleic acid sequence that is complementary to a second polynucleotide segment of the target mRNA and a detectable label or detectable tag, such as a fluorescent tag which is attached to the nucleic acid sequence. In some embodiments, the detectable tag is a fluorescent tag. In some embodiments, the detectable tag is a non-fluorescent tag. The fluorescent tag may be, for instance, a RNA specific fluorescent tag. In some embodiments, the fluorescent tag is selected from a 6-Carboxyfluorescein (6-FAM), 5-TAMRA, Cy3, Cy5, Fluorescein, TYE 563, TYE 664, TYE 705, and/or Yakima Yellow fluorescent tag. The detectable label may also be an RNA dye. In some embodiments, the fluorescent tag is a 6-Carboxyfluorescein (6-FAM). In some embodiments, the fluorescent tag is a 5-TAMRA. In some embodiments, the fluorescent tag is a Cy3. In some embodiments, the fluorescent tag is a Cy5. In some embodiments, the fluorescent tag is a Fluorescein. In some embodiments, the fluorescent tag is a TYE 563. In some embodiments, the fluorescent tag is a TYE 664. In some embodiments, the fluorescent tag is a TYE 705. In some embodiments, the fluorescent tag is a Yakima Yellow fluorescent tag. In some embodiments, the fluorescent tag is a RNA dye. In some embodiments, the second tag is a RNA dye.

In some embodiments, the fluorescent tag is a Cy5. In some embodiments, the fluorescent tag is a Fluorescein. In some embodiments, the fluorescent tag is a TYE 563. In some embodiments, the fluorescent tag is a TYE 664. In some embodiments, the fluorescent tag is a TYE 705. In some embodiments, the fluorescent tag is a Yakima Yellow fluorescent tag. In some embodiments, the fluorescent tag is a RNA dye. The detectable label of the second tag can produce a spectral signal that can be visualized.

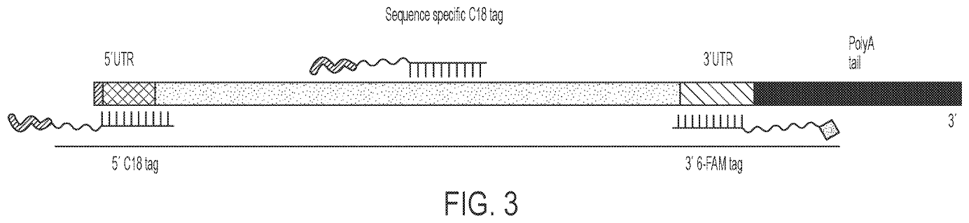

Thus, some embodiments, include a step of annealing in which a hydrophobic tag and a detectable tag hybridize to the target mRNA. The hydrophobic tag has a region that is complementary to and binds to a portion of the target mRNA and a hydrophobic region. A schematic of an exemplary tagged mRNA is shown in FIG. 3. In FIG. 3 the bar represents the mRNA and includes a 5′UTR, an ORF, a 3′UTR and a poly A tail. Three tags are depicted as bound to the mRNA. A 5′ C18 tag is attached to the 5′ UTR. A sequence specific C18 tag is attached to an internal sequence within the ORF. A 3′ 6-FAM tag is attached to the 3′UTR.

### Drag Tag

The tagged mRNA mixture can be exposed to a buffer having components capable of forming a drag tag when combined with the hydrophobic tag. A component capable of forming a drag tag may be, for instance, a lipophilic compound such as lipids or surfactants, having hydrophobic and hydrophilic portions. The specific type of lipophilic compound used will depend on the type of tag used. The lipophilic compound can interact with the hydrophobic region of the tag to produce a drag-tag. As used herein, the term “drag-tag” refers to a compound that modifies the charge-to-friction ratio of a molecule. The charge-to-friction ratio of a molecule can be modified by increasing the friction coefficient. When the mRNA is tagged and the tag forms a drag tag in the buffer solution, the lipophilic portion of the tagged molecule will serve to slow down the migration of the mRNA in an electric field when electrophoresis is performed. This leads to a greater separation of the nucleic acid components, allowing for significant advances in the determination of purity of that mixture or sample.

In some embodiments, the drag tag is any large molecule. In some embodiments, the drag tag is any large, uncharged molecule. In some embodiments the drag tag is protein(s), polymeric nanoparticles, and metal nanoparticles. In some embodiments the drag tag is protein(s). In some embodiments the drag tag is polymeric nanoparticles. In some embodiments the drag tag is metal nanoparticles.

A drag tag can form as a micelle, a liposome, or other structure. Liposomes may be formed for instance using a mixture of cholesterol and phospholipid such as dipalmitoylphosphatidylglycerol. In some embodiments, the drag tag is a micelle drag tag. Micelles may be formed, for instance, by adding a surfactant to a solution such as the buffer solution. The micelles can spontaneously form when the surfactant is exposed to the hydrophobic region. In some embodiments, the drag tag formed by the interaction of the hydrophobic tag and the surfactants may include a wormlike micelle, a spherical micelle (e.g., a circular triton micelle), polymeric micelles, supermicelles or a lamellar micelle. In some embodiments, the drag tag is selected from drag tag is selected from a circular triton micelle, a worm-like micelle, a spherical micelle, or a lamellar micelle. In some embodiments, the drag tag is a worm-like micelle. In some embodiments, the drag tag is a spherical micelle (e.g., a circular triton micelle). In some embodiments, the drag tag is polymeric micelles. In some embodiments, the drag tag is supermicelles. In some embodiments, the drag tag is a lamellar micelle. Typically, micelles will form if the concentration of the surfactant is in the range of 10⁻⁶ and 10⁻³ M. The surfactant can be non-ionic, cationic, anionic, zwitterionic, or combinations thereof.

In some embodiments, a surfactant, for instance in a buffer solution, is used to form the drag-tag. Non-ionic surfactants include, but are not limited to, acetylenic glycols, alkanolamides, alkanolamines, alkyl β-D-glycopyranosides, alkyl phenols, alkylglucosides, alkylmonoglucosides, fatty acids, fatty alcohols, fatty esters, glycerol esters, monododecyl ethers (such as C₁₂E₅, C₁₆E₆ and C₁₂E₈), phenol derivatives, poloxamers, poloxamines, polyoxyethylene acyl ethers, polyoxyethyleneglycol dodecyl ethers, sorbitols and sorbitan derivatives (such as Tween-20 and Tween-60), alkylphenol ethylene oxide condensates, alkyl ethylene oxide condensates, octylphenol ethylene oxide condensates (such as Triton X-100), fluoroalkylphenol ethylene oxide condensates, fluoroalkyl ethylene oxide condensates, partially fluorinated fluoroalkylphenol ethylene oxide condensates, partially fluorinated fluoroalkyl ethylene oxide condensates, fluorinated hydrocarbons (such as Cs F₁₅, C₁₀ F₁₉, C₁₂ F₂₃, and C₆ F₁₃), partially fluorinated hydrocarbons, fluorocarbon-based surfactants (such as Zonyl fluorosurfactants, Masurf FS-fluorosurfactants, Novec fluorosurfactants, and PEG-block-fluorocarbon copolymer fluorosurfactants), and combinations thereof. Cationic surfactants include, but are not limited to, alkylamines, quaternary amines, imidazolines, dialkylamine oxides, gemini surfactants, and combinations thereof. Anionic surfactants include, but are not limited to, salts of multiple acids, salts of fatty acids, sodium dodecyl sulfates, bile acid salts, isethionates, salts of tall oil acids, alcohol phosphates, inorganic phosphates, sarcosine derivatives, alcohol sulfates, alkyl phenol sulfates, sulfated triglycerides, alpha-olefin sulfonates, linear alkylbenzene sulfonates, aromatic sulfonates, sodium alkyl sulfonates, sulfosuccinates, taurates, gemini surfactants, and combinations thereof. Zwitterionic surfactants include, but are not limited to, amino acids, betaines, imidazolines, imino acids, phospholipids, gemini surfactants, and combinations thereof.

Based on the composition of the drag-tag, it will have a hydrodynamic radius. The drag tag increases the hydrodynamic drag of the mRNA, to which the tag is attached, during motion through a liquid substance such as during electrophoresis, with or without the presence of an electroosmotic flow. In some embodiments, drag tags that induce a significant amount of hydrodynamic friction can be used to enhance the electrophoretic separation process. For example, drag tags with significant hydrodynamic friction may permit greater separation of a large quantity of nucleic acid sizes, in addition to separating nucleic acids of the same or similar sizes. When applied to an IVT mixture containing RNA of multiple sizes or a mixed mRNA composition having multiple mRNAs (of the same or different sizes), it is possible to achieve greater nucleotide resolution and/or increased read lengths. The tight bonding between the hydrophobic region and the surfactant to form the drag-tag results in the drag-tags being moved with the target mRNA a distance dependent upon the hydrodynamic radius of each individual drag-tag. Thus, the drag-tags can be separated by their hydrodynamic radius. Generally, drag-tags in an aqueous suspension with hydrodynamic radius between 1 nm and 1,000 nm can be assayed using the methods.

### Detection Methods and Buffer

The tagged mRNA molecules can be separated using separation techniques such as electrophoresis. The hydrophobic region of the tag can form a drag tag in a capillary electrophoresis assay. The presence of the target mRNA of interest can then be determined by detecting the detectable tag and mobility of the target mRNA/drag tag complex compared to other components in the mixture. It has been demonstrated herein that in a complex mixture of RNAs, it is possible to attach a sequence-specific tag, form a drag-tag, and separate out only that one RNA sequence without affecting the other RNAs. The application of these methods and compositions for detecting a target mRNA in a mixture have broad applications, such as determining the purity of target mRNA in a mixture for mRNA vaccine or mRNA therapeutic manufacturing. The Examples demonstrate that as a target mRNA degrades the output signal decreases. For example, when a sample having four RNAs experienced degradation similar degradation was observed in all of them. The more degraded the RNA, the less signal was observed in the full-length peak.

The buffer, which may be the same buffer used to form the drag tags with surfactants, may be used to in the electrophoresis method. The buffer can comprise other buffer components such as a buffering system in addition to the surfactant. The buffering system may include, for instance, tris(hydroxymethyl)aminomethane (“Tris”) acetate, Tris HCl, Tris-2-(N-morpholino)ethanesulfonic ac (MES), phosphate buffered saline, Tris-acetate-EDTA (TAE) buffer, or sodium chloride, and/or combination(s) thereof. In some embodiments, the buffer solution used in CE may be a specific pH. In some embodiments, the pH of the buffer solution may be a neutral, basic, or acidic pH. In some embodiments, the pH of the buffer solution may be 6. In some embodiments, the pH of the buffer solution may be 7. In some embodiments, the pH of the buffer solution may be 8. In some embodiments, the pH of the buffer solution may be between 1-6 (e.g., 1-6, 1-5, 1-4, 1-3, 2-6, 2-5, 2-4, 2-3, 3-6, 3-5, 3-4, 4-6, 4-5, 5-6). In some embodiments, the pH of the buffer solution is about 1. In some embodiments, the pH of the buffer solution is about 2. In some embodiments, the pH of the buffer solution is about 3. In some embodiments, the pH of the buffer solution is about 4. In some embodiments, the pH of the buffer solution is about 5. In some embodiments, the pH of the buffer solution may be between 6-8 (e.g., 6-8, 6-7, 7-8). In some embodiments, the pH of the buffer solution may be between 8-14 (e.g., 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, 13-14). In some embodiments, the pH of the buffer solution is about 9. In some embodiments, the pH of the buffer solution is about 10. In some embodiments, the pH of the buffer solution is about 11. In some embodiments, the pH of the buffer solution is about 12. In some embodiments, the pH of the buffer solution is about 13. In some embodiments, the pH of the buffer solution is about 14.

Some aspects provide methods of detecting a target mRNA in a mixture comprising attaching a first tag and a second tag to a mRNA molecule to generate a tagged mRNA molecule. The first tag may comprise a hydrophobic region. The second tag may be a tag detectable by a detector. It should be understood to one of ordinary skill in the art that the first tag and the second tag may be reversed (e.g., the first tag is detectable by a detector and the second tag has a hydrophobic region). The tagged target mRNA can be separated from other components of a mixture using separation methods. The separation methods may be any method that involves mobility or charge based separation. For instance, the separation methods may be electrophoresis, such as, for example, free solution capillary electrophoresis (CE), microchip electrophoresis, or free flow electrophoresis. In some embodiments, the separation methods are free solution capillary electrophoresis methods. As described herein, the type of CE employed may be selected from the group consisting of end-labeled free solution electrophoresis (ELFSE), micellar end-labeled free solution electrophoresis (miELFSE), micellular electrokinetic chromatography, microemulsion electrokinetic chromatography, liposome electrokinetic chromatography, and capillary electrophoresis. In some embodiments, the free solution capillary electrophoresis is end-labeled free solution electrophoresis (ELFSE). In some embodiments, the free solution capillary electrophoresis is micellar end-labeled free solution electrophoresis (miELFSE). In some embodiments, the free solution capillary electrophoresis is micellular electrokinetic chromatography. In some embodiments, the free solution capillary electrophoresis is microemulsion electrokinetic chromatography. In some embodiments, the free solution capillary electrophoresis is liposome electrokinetic chromatography. In some embodiments, the free solution capillary electrophoresis is capillary electrophoresis.

As described herein, capillary electrophoresis, or CE, is a technique which separates biomolecules on a capillary tube using a liquid or gel polymer medium. In capillary electrophoresis, separation is done inside a capillary tube and liquid polymers may be used. In CE detection is done through spectrophotometric automated detectors.

CE separates ions based on electrophoretic mobility by applying voltage. Electrophoretic mobility can be affected by the charge of the molecule, the viscosity of the buffer, and the actual size of the atom. End-labeled free solution electrophoresis (ELFSE) decreases the electrophoretic mobility of a nucleic acid by attaching a drag inducing entity (i.e., a “drag tag”) to the nucleic acid. Therefore, a drag tag/nucleic acid complex may be separated from other products and their degradant.

In some embodiments, CE may be performed using a capillary. In some embodiments, the capillary used may be chosen from the following group: silica capillary and pre-coated capillary. For example, a pre-coated capillary may be NCHO capillary. NCHO capillary is a silica capillary covalently coated with PVA. Unmodified bare fused silica capillary is useful in other embodiments. Capillary coatings can be covalently coated or dynamically coated (usually charge associated). Polyvinylalcohol, polyethylene oxide/polyethylene glycol, fluorinated polymer are covalent coatings useful in some embodiments.

The output of the electrophoresis can be an electropherogram which represents the separation of the RNA in the buffer.

In some embodiments, CE may be performed at different temperatures. In some embodiments, the temperature is about 20° C. In some embodiments, the temperature CE may be performed at may be within a range from 20-25 (e.g., 20-25, 20-24, 20-23, 20-22, 20-21, 21-25, 21-24, 21-23, 21-22, 22-25, 22-24, 22-23, 23-25, 23-24, 24-25° C.) In some embodiments, the temperature is about 21° C., 22° C., 23° C., 24° C., 25° C. In some embodiments, the temperature is about 21° C. In some embodiments, the temperature is about 22° C. In some embodiments, the temperature is about 23° C. In some embodiments, the temperature is about 24° C. In some embodiments, the temperature is about 25° C. In some embodiments, the temperature CE may be performed at is within a range from 25-35 (e.g., 25-35, 25-30, 30-35° C.). In some embodiments, the temperature CE may be performed at may be within a range from 25° C. to 35° C. In some embodiments, the temperature is about 25° C., 26° C., 27° C., 28° C., 29° C., 30° C., 31° C., 32° C., 33° C., 34° C., 35° C. In some embodiments, the temperature is about 26° C. In some embodiments, the temperature is about 27° C. In some embodiments, the temperature is about 28° C. In some embodiments, the temperature is about 29° C. In some embodiments, the temperature is about 30° C. In some embodiments, the temperature is about 31° C. In some embodiments, the temperature is about 32° C. In some embodiments, the temperature is about 33° C. In some embodiments, the temperature is about 34° C. In some embodiments, the temperature is about 35° C. In some embodiments, the temperature CE may be performed at is within a range from 30-40 (e.g., 30-40, 30-35, 35-40° C.) In some embodiments, the temperature CE may be performed at may be within a range from 30° C. to 40° C. In some embodiments, the temperature is about 30° C., 31° C., 32° C., 33° C., 34° C., 35° C., 36° C., 37° C., 38° C., 39° C., 30° C. In some embodiments, the temperature is about 36° C. In some embodiments, the temperature is about 37° C. In some embodiments, the temperature is about 38° C. In some embodiments, the temperature is about 39° C. In some embodiments, the temperature is about 40° C.

In some embodiments, CE may be performed using a buffer solution, wherein the buffer solution is comprised of a plurality of reagents comprising a first surfactant and a second surfactant. In some embodiments, buffer solution is comprised a first surfactant and a second surfactant. In some embodiments, buffer solution is comprised a first surfactant. In some embodiments, buffer solution is comprised a second surfactant. The surfactant maybe be, for instance, any of the surfactants disclosed herein. In some embodiments, the first surfactant is pentaethylene glycol monododecyl ether and the second surfactant is pentaethylene glycol monodecyl ether. It should be understood to one of ordinary skill in the art that the first surfactant and the second surfactant may be reversed (e.g., the first surfactant is pentaethylene glycol monodecyl ether and the second surfactant is pentaethylene glycol monododecyl ether). In some embodiments, the first surfactant is pentaethylene glycol monododecyl ether. In some embodiments, the second surfactant is pentaethylene glycol monododecyl ether. In some embodiments, the first surfactant and the second surfactant may be collectively referred to herein as “the surfactants”. In some embodiments, the surfactants may be provided in different ratios. In some embodiments, the ratio of pentaethylene glycol monododecyl ether to pentaethylene glycol monodecyl ether may be 8:1. In some embodiments, a CiEj buffer may be used. As described herein, CiEj buffer refers to a mixture of surfactants. In some embodiments, the surfactants include, but are not limited to polyoxyethylene glycol alkyl ether amphiphiles, Triton X-100, pentaethylene glycol monododecyl ether (C₁₂E₅), pentaethylene glycol monodecyl ether (C₁₀E₅), and/or urea.

In some embodiments, the CiEj buffer includes about 30-60, 30-55, 30-50, 30-45, 30-40, 30-35, 35-60, 35-55, 35-50, 35-45, 35-40, 40-60, 40-55, 40-50, 40-45, 45-60, 45-55, 45-50, 48-60, 48-55, 48-50, 50-60, 50-55, or 55-60 mM pentaethylene glycol monododecyl ether (C₁₂E₅). In some embodiments, the CiEj buffer includes about 30 mM, 35 mM, 40 mM, 45 mM, 48 mM, 50 mM, 55 mM, 60 mM pentaethylene glycol monododecyl ether (C₁₂E₅). In some embodiments, the CiEj buffer includes about 30 mM pentaethylene glycol monododecyl ether (C₁₂E₅). In some embodiments, the CiEj buffer includes about 35 mM (C₁₂E₅). In some embodiments, the CiEj buffer includes about 40 mM (C₁₂E₅). In some embodiments, the CiEj buffer includes about 45 mM (C₁₂E₅). In some embodiments, the CiEj buffer includes about 50 mM (C₁₂E₅). In some embodiments, the CiEj buffer includes about 55 mM (C₁₂E₅). In some embodiments, the CiEj buffer includes about 60 mM (C₁₂E₅). In some embodiments, the CiEj buffer includes about 48 mM pentaethylene glycol monododecyl ether (C₁₂E₅).

In some embodiments, the CiEj buffer includes about 5-20 (e.g., 5-20, 5-15, 5-10, 6-20, 6-15, 6-10, 10-20, 10-15, 15-20) mM pentaethylene glycol monodecyl ether (C₁₀E₅). In some embodiments, the CiEj buffer includes about 1, 2, 3, 4, 5, 6, 7, 8, 9, 10, 11, 12, 15, 20 mM pentaethylene glycol monodecyl ether (C₁₀E₅). In some embodiments, the CiEj buffer includes about 1 mM pentaethylene glycol monodecyl ether (C₁₀E₅). In some embodiments, the CiEj buffer includes about 2 mM pentaethylene glycol monodecyl ether (C₁₀E₅). In some embodiments, the CiEj buffer includes about 3 mM pentaethylene glycol monodecyl ether (C₁₀E₅). In some embodiments, the CiEj buffer includes about 4 mM pentaethylene glycol monodecyl ether (C₁₀E₅). In some embodiments, the CiEj buffer includes about 5 mM pentaethylene glycol monodecyl ether (C₁₀E₅). In some embodiments, the CiEj buffer includes about 6 mM pentaethylene glycol monodecyl ether (C₁₀E₅). In some embodiments, the CiEj buffer includes about 7 mM pentaethylene glycol monodecyl ether (C₁₀E₅). In some embodiments, the CiEj buffer includes about 8 mM pentaethylene glycol monodecyl ether (C₁₀E₅). In some embodiments, the CiEj buffer includes about 9 mM pentaethylene glycol monodecyl ether (C₁₀E₅). In some embodiments, the CiEj buffer includes about 10 mM pentaethylene glycol monodecyl ether (C₁₀E₅). In some embodiments, the CiEj buffer includes about 11 mM pentaethylene glycol monodecyl ether (C₁₀E₅). In some embodiments, the CiEj buffer includes about 12 mM pentaethylene glycol monodecyl ether (C₁₀E₅). In some embodiments, the CiEj buffer includes about 15 mM pentaethylene glycol monodecyl ether (C₁₀E₅). In some embodiments, the CiEj buffer includes about 20 mM pentaethylene glycol monodecyl ether (C₁₀E₅). In some embodiments, the CiEj buffer includes about 6 mM pentaethylene glycol monodecyl ether (C₁₀E₅).

In some embodiments, the CiEj buffer includes (polyoxyethylene glycol alkyl ether amphiphiles) Triton X-100, 48 mM pentaethylene glycol monododecyl ether (C₁₂E₅), and 6 mM pentaethylene glycol monodecyl ether (C₁₀E₅), and with 3M urea. In some embodiments, mixtures containing 0M to 1M urea may be used. In some embodiments, 1M to 3M urea may be used. In some embodiments, 3M to 5M urea may be used.

In some embodiments, the tagged mRNA is added to a buffer solution. In some embodiments, the buffer solution is a CiEJ buffer solution. In some embodiments, the buffer solution is a CiEJ buffer solution containing urea. In some embodiments, the buffer solution interacts with the first tag and/or the second tag. In some embodiments, the buffer solution interacts with the first tag. In some embodiments, the buffer solution interacts with the second tag. In some embodiments, the buffer solution interacts with the first tag and/or the second tag, optionally the hydrophobic region of the first tag and/or the second tag to form a drag tag. In some embodiments, the buffer solution interacts with the first tag forms a drag tag. In some embodiments, the buffer solution interacts with the second tag forms a drag tag. In some embodiments, the buffer solution interacts with the first tag and the second tag to form a drag tag.

If the nucleic acids in the mixture form any secondary structures, the mixtures can be treated to minimize those structures. For instance, a denaturing agent may be included with the buffer or separately added to the mixture. In some embodiments, a mild denaturing agent such as urea may be used to avoid disrupting the hybridized tag sequences. By adding urea to the buffer, it has been demonstrated that the presence of RNA secondary structure and any RNA-RNA interactions could be minimized without detaching the DNA (e.g., LNA/2′OMe) tags.

In some embodiments, the mRNA tagged with a hydrophobic tag and a detectable tag may be introduced to CE using an injection method. In some embodiments, the tagged mRNA is added to the buffer solution by an injection method. The injection method may be chosen from an electrokinetic injection or a pressure injection. In some embodiments, the injection method is a pressure injection. In some embodiments, the injection method is an electrokinetic injection.

In some embodiments, the CE may be coupled to a detector which may be used to detect a signal corresponding to the target mRNA/drag tag complex, and therefore identifying a signal corresponding to the target mRNA.

The methods can be useful for determining purity of and/or quantitating a target mRNA in a mixture. As described herein, a “mixture” refers to a composition containing a target mRNA of interest, that may also contain RNA fragments, truncated RNA, other nucleotide sequences, and other background impurities. In some embodiments, a mixture may be the output from an In Vitro Transcription (IVT) reaction. In other embodiments, the mixture may be a complex solution such as a final drug product. In some embodiments, the mixture may be, for instance, a liquid sample, such as reaction sample, a biological sample, a pharmaceutical product sample, etc.

### In Vitro Transcription (IVT)

Some aspects relate to mRNAs produced by “in vitro transcription” or IVT. When mRNA is produced in an IVT reaction, the resultant product includes full length mRNA and fragments thereof. It is important for research as well as clinical, therapeutic analysis methods to determine the purity of full-length mRNA and the methods disclosed herein provide improved methods of detecting purity. IVT methods produce (e.g., synthesize) an RNA transcript (e.g., mRNA transcript) by contacting a DNA template (e.g., a first input DNA and a second input DNA) with an RNA polymerase (e.g., a T7 RNA polymerase, a T7 RNA polymerase variant, etc.) under conditions that result in the production of the RNA transcript. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction. Some embodiments, comprise methods of detecting mRNA purity in a IVT sample.

In some embodiments, a wild-type T7 polymerase is used in an IVT reaction. In some embodiments, a modified or mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference. In some embodiments, the RNA polymerase (e.g., T7 RNA polymerase or T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

### Drug Product

Also provided are methods of detecting mRNA purity in a pharmaceutical sample such as a drug product. In some embodiments, a drug product comprises a lipid nanoparticle comprising an ionizable lipid, a structural lipid, a phospholipid, and the target mRNA. In some embodiments, the LNP comprises an ionizable lipid, a PEG-modified lipid, a phospholipid and a structural lipid.

In some embodiments, the mRNA drug product comprises a single target mRNA in an LNP. The methods disclosed herein may be used detect the target mRNA and/or determine purity or quantitate the mRNA in the LNP. For example, in some embodiments, the method may be performed directly on the drug product. In some embodiments, the drug product may be solubilized before analysis. Solubilization, for instance, with Triton, is used to liberate the RNA from the lipids of the drug product. The lipids in some embodiments, may be extracted with IPA such that they are removed from the mRNA.

Certain mRNA drug products can include multiple mRNAs and the methods disclosed herein can be used to determine the presence of those different mRNAs in mixtures such as RNA samples or drug products. In some embodiments, the mixtures comprise more than 1 mRNA of similar sizes or the same size. mRNAs have similar sizes if the mRNAs are within about 100 nucleotides of one another. mRNAs of different sizes have a size differential of greater than 100 nucleotides. In some embodiments, the mixtures comprise about 2-50, 2-45, 2-40, 2-35, 2-30, 2-25, 2-20, 2-15, 2-14, 2-13, 2-12, 2-11, 2-10, 2-9, 2-8, 2-7, 2-6, 2-5, 2-4, 2-3, 3-50, 3-45, 3-40, 3-35, 3-30, 3-25, 3-20, 3-15, 3-14, 3-13, 3-12, 3-11, 3-10, 3-9, 3-8, 3-7, 3-6, 3-5, 3-4, 4-50, 4-45, 4-40, 4-35, 4-30, 4-25, 4-20, 4-15, 4-14, 4-13, 4-12, 4-11, 4-10, 4-9, 4-8, 4-7, 4-6, 4-5, 5-50, 5-45, 5-40, 5-35, 5-30, 5-25, 5-20, 5-15, 5-14, 5-13, 5-12, 5-11, 5-10, 5-9, 5-8, 5-7, 5-6, 2, 6-20, 6-15, 6-14, 6-13, 6-12, 6-11, 6-10, 6-9, 6-8, 6-7, 7-20, 7-15, 7-14, 7-13, 7-12, 7-11, 7-10, 7-9, 7-8, 8-20, 8-15, 8-14, 8-13, 8-12, 8-11, 8-10, 8-9, 9-20, 9-15, 9-14, 9-13, 9-12, 9-11, 9-10, 10-20, 10-15, 10-14, 10-13, 10-12, or 10-11 mRNA of similar sizes or different sizes or the same size or a mixture of similar, different and/or the same size.

Aspects of the disclosure relate to populations of molecules. As used herein, a “population” of RNA molecules generally refers to a preparation comprising a plurality of copies of the molecule (e.g., mRNA) of interest. In some embodiments, a population is a homogenous population comprising a single mRNA species. As used herein, an mRNA species refers to an mRNA molecule having a given nucleotide sequence. Two or more mRNA molecules having identical nucleotide sequences and backbone compositions belong to the same mRNA species, while two mRNA molecules having different nucleotide sequences and/or different backbone compositions belong to different mRNA species. In some embodiments, a population is a heterogenous population comprising two or more mRNA species. In some embodiments, a heterogenous population comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, or more mRNA species.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos.: WO2017075531, WO2015199952, WO2013086354, or WO2013116126, or selected from formulae CLI-CLXXXXII of U.S. Pat. No. 7,404,969; each of which is hereby incorporated by reference in its entirety for this purpose.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In some embodiments, the ionizable amino lipid may have a positively charged hydrophilic head and a hydrophobic tail that are connected via a linker structure. In addition to these, an ionizable lipid may also be a lipid including a cyclic amine group.

### Unique Codes

In some embodiments, mRNAs of a multivalent mRNA composition are detected and/or purified according to the methods disclosed herein. In some embodiments, multivalent RNA compositions may further comprise unique codes. In some embodiments, the mRNA unique codes are used to identify the presence of mRNA or determine a relative ratio of different mRNAs in a mixture (e.g., a reaction product or a drug product), using routine methods for identifying unique sequences. In some embodiments, the unique codes may also serve as a template sequence for the nucleic acid sequence of the tags disclosed herein. In some embodiments, the unique codes serve both functions.

Thus, aspects of the disclosure relate to methods of determining purity of mRNA compositions, wherein the target mRNA comprises one or more (e.g., 1, 2, 3, 4, or more) unique identifier sequences or unique code sequences. As used herein, an “identifier sequence” or “unique code sequence” refers to a sequence of a biological molecule (e.g., nucleic acid) that when combined with the sequence of another biological molecule serves to identify the other biological molecule. Typically, a unique code sequence is a heterologous sequence that is incorporated within or appended to a sequence of a target biological molecule and utilized as a reference to identify a target molecule of interest. In some embodiments, a unique code sequence is a sequence of a nucleic acid (e.g., a heterologous or synthetic nucleic acid) that is incorporated within or appended to a target nucleic acid and utilized as a reference to identify the target nucleic acid. In some embodiments, a unique code sequence is of the formula (N) n. In some embodiments, n is an integer in the range of 5 to 20, 5 to 10, 10 to 20, 7 to 20, or 7 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, N are each nucleotides that are independently selected from A, G, T, U, and C, or analogues thereof.

In some embodiments, one or more RNA species (e.g., RNA of a given sequence) of a RNA composition (e.g., a multivalent RNA composition) comprises a distinct unique codes. Unique codes may differ in sequence length, base composition, or sequence length and base composition. In some embodiments, each RNA species in a multivalent RNA composition comprises a unique code that differs from the unique code of every other mRNA in the multivalent RNA composition. In some embodiments, each RNA species in a multivalent RNA composition comprises a unique code with a different length. In some embodiments, each RNA species in a multivalent RNA composition comprises a unique code with length between 0 and 100, 0 and 50, 0 and 30, 0 and 20, 0 and 10, or 0 and 5 nucleotides. In some embodiments, each RNA species in a multivalent RNA composition comprises a unique code with length between 1 and 100, 1 and 50, 1 and 30, 1 and 20, 1 and 10, or 1 and 5 nucleotides.

In some embodiments, one or more in vitro transcribed mRNAs comprise one or more unique code sequences in an untranslated region (UTR), such as a 5′ UTR or 3′ UTR. Inclusion of a unique code sequence in the UTR of an mRNA prevents the unique code sequence from being translated into a peptide. In some embodiments, inclusion of a unique code in a UTR does not negatively affect the translation of (e.g., reduce translation of) the mRNA into a protein. In some embodiments, a unique code sequence is positioned in a 3′ UTR of an mRNA. In some embodiments, the unique code sequence is positioned upstream of the poly A tail of the mRNA. In some embodiments, the unique code sequence is positioned downstream of (e.g., after) the polyA tail of the mRNA. In some embodiments, the unique code sequence is positioned between the last codon of the ORF of the mRNA and the first “A” of the polyA tail of the mRNA. In some embodiments, a polynucleotide unique code positioned in a UTR comprises between 1 and 30 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides).

Exemplary unique code sequences include: GGAA, GGUUA, GACCA, GGACCA, GGCCAAA, GGCCAAGA, GGCCAAGGA, CCCGUACCCCC (SEQ ID NO: 12), AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; UGACCA; GACCUACGA; CCAA; GUUA; CCUUA; AGACC; UUACCA; GGAGGA; GUACGGA; GUUCAUU; GGCUUCUGACCA (SEQ ID NO: 1); GGCCACUCGUUAAGA (SEQ ID NO: 2); GGCCACUGAAGCCAUUGAAG (SEQ ID NO:3); GGCCACUGAAGCCAUUGUCAAGGA (SEQ ID NO: 4); GGCCACUGAAGCCAUUGUCACCGAA (SEQ ID NO: 5); GGCGAAGCACUCGUGGCCAUUCGCA (SEQ ID NO: 6); GGCCAAGGA; GGCCAAGGAA (SEQ ID NO: 7); GGCCAAGGAAA (SEQ ID NO: 8); GGCCACUGAAGA (SEQ ID NO: 9); GGCCACUGAAGCCAUU (SEQ ID NO: 10); or GGCCACUGAAGGAAG (SEQ ID NO: 11).

Nucleic Acids/Target mRNA

Aspects of the disclosure relate to methods of detecting a target mRNA in a mixture, which may comprise, in addition to the target mRNA, additional mRNAs, RNA fragments and other nucleic acids. As used herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. A nucleic acid (e.g., mRNA) may include a substitution and/or modification. In some embodiments, the substitution and/or modification is in one or more bases and/or sugars. For example, in some embodiments, a nucleic acid (e.g., mRNA) includes nucleic acids having backbone sugars that are attached, i.e., covalently attached to low molecular weight organic groups other than a hydroxyl group at the 2′ position and other than a phosphate group or hydroxy group at the 5′ position. Thus, in some embodiments, a substituted or modified nucleic acid (e.g., mRNA) includes a 2′-O-alkylated ribose group. In some embodiments, a modified nucleic acid (e.g., mRNA) includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid (e.g., mRNA) is heterogeneous in backbone composition thereby containing any possible combination of polymer units linked together.

As described herein, a “mRNA” refers to messenger ribonucleic acid (mRNA), which is any ribonucleic acid (RNA) that encodes a (at least one) protein (a naturally occurring, non-naturally occurring, or modified polymer of amino acids) and can be translated to produce the encoded protein in vitro, in vivo, in situ, or ex vivo. In some embodiments, the mRNA may comprise one or more RNAs, each having an open reading frame (ORF). In some embodiments, each RNA (e.g., mRNA) further comprises a 5′ UTR, 3′ UTR, a poly(A) tail and/or a 5′ cap analog. It should also be understood that the mRNA of the present disclosure may include any 5′ untranslated region (UTR) and/or any 3′ UTR.

An open reading frame (ORF) is a continuous stretch of deoxyribonucleic acid (DNA) or RNA beginning with a start codon (e.g., methionine (ATG or AUG)) and ending with a stop codon (e.g., TAA, TAG or TGA, or UAA, UAG or UGA). An ORF typically encodes a protein. A protein may be an antigen such as a vaccine antigen or therapeutic or diagnostic protein. As used herein, a “vaccine antigen” is a biological preparation that improves immunity to a particular disease or infectious agent. Vaccine antigens encoded by an mRNA described herein may be utilized to treat conditions or diseases in many therapeutic areas such as, but not limited to, cancer, allergy, and infectious disease. In some embodiments, the cancer vaccines may be personalized cancer vaccines in the form of a concatemer or individual RNAs encoding peptide epitopes or a combination thereof.

A target mRNA is an mRNA present in a mixture which will be detected. A mixture may include more than one mRNA. However, the mRNA that will be detected according to the methods disclosed herein is a target mRNA. When the mixture includes more than one mRNA to be detected, multiple target mRNAs are present.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved.

Modified nucleotides may include modified nucleobases. For example, a target mRNA provided herein may include a modified nucleobase selected from pseudouridine (ψ), 1-methylpseudouridine (m1ψ), 1-methylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an mRNA includes a combination of at least two (e.g., 2, 3, 4 or more) of the foregoing modified nucleobases.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators). Such isolated molecules are those that are separated from their natural environment.

### Untranslated Regions

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the target mRNAs as flanking regions to an ORF. This publication is incorporated by reference herein for this purpose.

Additional exemplary UTRs that may be utilized in the nucleic acids provided herein include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H (+)-ATP synthase (β-mRNA) 3′ UTR; a GLUT1 3′ UTR; a MEF2A 3′ UTR; a B-F1-ATPase 3′ UTR; functional fragments thereof and combinations thereof.

In some embodiments, the nucleic acid may comprise multiple UTRs, e.g., a double, a triple or a quadruple 5′ UTR or 3′ UTR.

### Poly(A) Tails

Some aspects relate to methods of detecting target mRNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

### EXAMPLES

### Example 1: Mobility Based Separation of mRNA-1

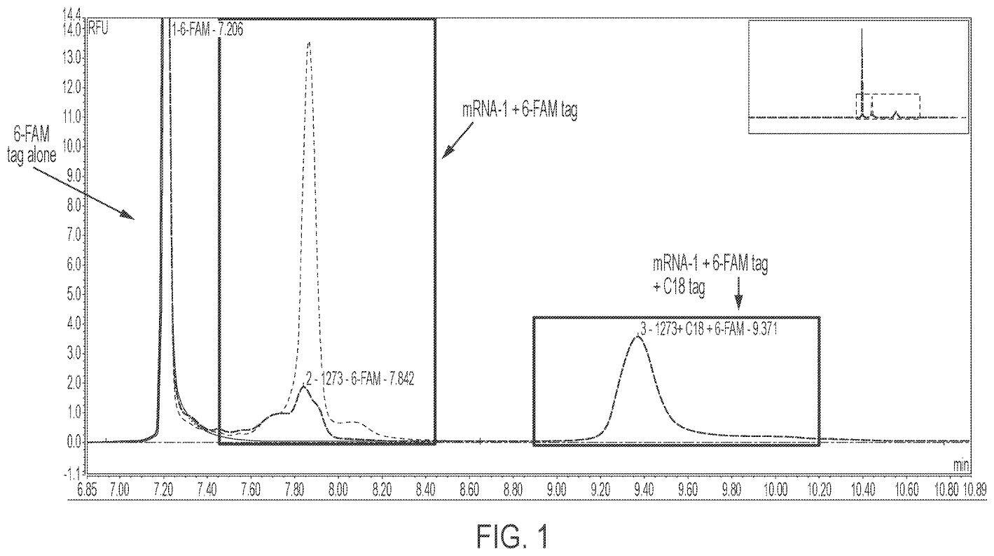

Methods of detecting the relative amount of target mRNA in a mixture compared to the total amount of RNA may provide information on how pure a mixture is. To achieve mobility-based separation of a target mRNA, mRNA-1 was detected in a sample mixture as follows. A mixture containing a target mRNA comprised of a 5′ untranslated region (UTR), an open reading frame (ORF), a 3′ UTR and a polyA tail was subjected to an annealing procedure to attach a 6-FAM detectable tag and a C18 hydrophobic tag to the 3′ UTR and 5′ UTR respectively. The target mRNA with the 6-FAM and C18 hydrophobic tags were subjected to capillary electrophoresis using an NCHO capillary at 20° C. in CiEj buffer. The resulting electropherogram, FIG. 1, shows three distinct peaks. In order of increasing mobility, there is a first peak representative of the detectable tag alone, 6-FAM, a second peak representative of mRNA-1 and the 6-FAM detectable tag, and a third peak representative of mRNA-1, the 6-FAM detectable tag, and the C18 hydrophobic tag. This example illustrates that mobility-based separation of a target mRNA and a drag tag may be achieved.

### Example 2: Target Specificity of Method

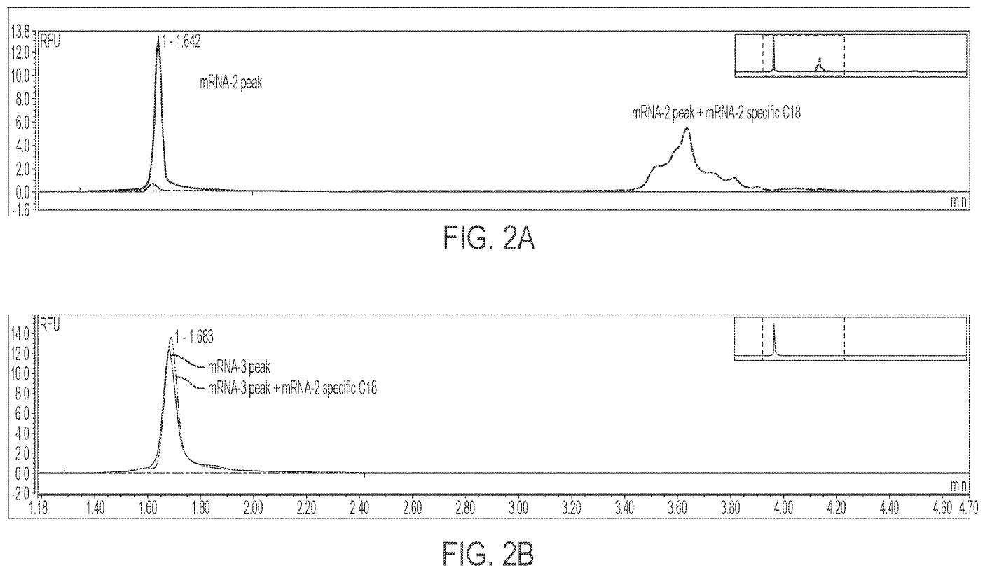

Methods of detecting the relative amount of target mRNA in a mixture compared to the amount of other mRNAs or total amount of RNA may provide information on how pure a mixture is. To demonstrate sequence-specific separation of a target mRNA based on a sequence specific hydrophobic tag, two mRNA constructs (mRNA-2 and mRNA-3, both of which contain a 5′ untranslated region (UTR), an open reading frame (ORF), a 3′ UTR and a polyA tail) were each detected in the presence of a hydrophobic tag specific for mRNA-2. More specifically, capillary electrophoresis was conducted with mRNA-2 with and without an annealed C18 hydrophobic tag specific to mRNA-2. The resulting electropherograms show that the non-tagged mRNA-2 can be distinguished from tagged mRNA-2 (see FIG. 2A). Capillary electrophoresis was also conducted with mRNA-3 with and without a C18 hydrophobic tag specific for mRNA-2. As shown in FIG. 2B, the tag did not successfully anneal to the mRNA-3, and so the mRNA-3 peaks overlapped in the presence and absence of the tag. This example illustrates that sequence specific separation of a target mRNA from non-target mRNA may be achieved using the methods described herein.

### FURTHER EMBODIMENTS

Embodiment 1: A method for detecting a target mRNA, the method comprising:
(a) attaching a first tag and a second tag to a mixture comprising a target mRNA molecule to generate a tagged mRNA molecule,
(b) subjecting the tagged mRNA molecule to a capillary electrophoresis assay, wherein the first tag causes a change in separation properties of the mRNA molecule in the assay to separate the mRNA molecule from other components of the mixture, and
(c) detecting a signal corresponding to the second tag based on the separated mRNA molecule, and thereby identifying a signal corresponding to the target mRNA.

Embodiment 2: The method of embodiment 1, wherein the capillary electrophoresis assay is an end labeled free solution electrophoresis (ELFSE) assay.

Embodiment 3: The method of embodiment 1, wherein the capillary electrophoresis assay is a micellar end labeled free solution electrophoresis (miELFSE) assay.

Embodiment 4: The method of embodiment 1, wherein the first tag comprises a nucleic acid sequence that is complementary to a polynucleotide segment of the target mRNA in the 5′ UTR.

Embodiment 5: The method of embodiment 1, wherein the first tag comprises a nucleic acid sequence that is complementary to a polynucleotide segment of the target mRNA in the 3′ UTR.

Embodiment 6: The method of embodiment 5, wherein the first tag comprises a hydrophobic region.

Embodiment 7: The method of embodiment 6, wherein the hydrophobic region is a C18 molecule, DNA base modification, LNA base modification or 2′ Ome base modification.

Embodiment 8: The method of embodiment 1, wherein the hydrophobic region is a C18 molecule.

Embodiment 9: The method of embodiment 1 or 5, wherein the tagged RNA is introduced to a buffer solution.

Embodiment 10: The method of embodiment 9, wherein the buffer solution interacts with the hydrophobic region to form a drag tag.

Embodiment 11: The method of embodiment 1, wherein the second tag comprises a nucleic acid sequence that is complementary to a polynucleotide segment of the target mRNA in the 3′ UTR.

Embodiment 12: The method of embodiment 1, wherein the second tag comprises a nucleic acid sequence that is complementary to a polynucleotide segment of the target mRNA in the 5′ UTR.

Embodiment 13: The method of embodiment 1, wherein the second tag comprises a fluorescent tag attached to the nucleic acid sequence.

Embodiment 14: The method of embodiment 13, wherein the fluorescent tag is a 6-FAM fluorescent tag.

Embodiment 15: The method of embodiment 13, wherein the second tag comprises an RNA dye.

Embodiment 16: The method of embodiment 1, wherein the second tag has a hydrophobic region.

Embodiment 17: The method of embodiment 1, wherein the first tag and the second tag are attached to the target mRNA using an annealing procedure.

Embodiment 18: The method of embodiment 17, wherein the annealing procedure comprises incubating the target mRNA with the first tag and the second tag at 75° C. with 25 mM KCl in TE.

Embodiment 19: The method of embodiment 9, wherein the tagged mRNA is introduced to the buffer solution by an injection method.

Embodiment 20: The method of embodiment 19, wherein the injection method is a pressure injection.

Embodiment 21: The method of embodiment 19, wherein the injection method is an electrokinetic injection.

Embodiment 22: The method of embodiment 9, wherein the buffer solution comprises a plurality of reagents comprising a first surfactant and a second surfactant.

Embodiment 23: The method of embodiment 22, wherein the buffer solution is a CiEJ buffer solution containing or not containing urea.

Embodiment 24: The method of embodiment 22, wherein the first surfactant is pentaethylene glycol monododecyl ether.

Embodiment 25: The method of embodiment 22, wherein the second surfactant is pentaethylene glycol monodecyl ether.

Embodiment 26: The method of embodiment 22, wherein the first surfactant is pentaethylene glycol monodecyl ether.

Embodiment 27: The method of embodiment 22, wherein the second surfactant is pentaethylene glycol monododecyl ether.

Embodiment 28: The method of embodiment 10, wherein the drag tag is a micelle drag tag.

Embodiment 29: The method of embodiment 10, wherein the drag tag is selected from a circular triton micelle, a worm-like micelle, a spherical micelle or a lamellar micelle.

Embodiment 30: The method of embodiment 10, wherein the drag tag is a cylindrical worm-like micelle.

Embodiment 31: The method of embodiment 1, wherein the signal corresponding to the target mRNA is based on a mobility-base separation.

Embodiment 32: The method of embodiment 1, wherein the target mRNA is greater than 500 nucleotides.

Embodiment 33: The method of embodiment 1, wherein the target mRNA is 500-15,000 nucleotides, 500-12,000 nucleotides, 500-10,000 nucleotides, 500-8,000 nucleotides, 1,000-15,000 nucleotides, 1,000-12,000 nucleotides, 1,000-10,000 nucleotides or 1,000-8,000 nucleotides.

Embodiment 34: A method for detecting a mRNA in a mixture, the method comprising:
subjecting a liquid sample comprising the mRNA to free solution electrophoresis, wherein the mRNA comprises a first tag that increases hydrodynamic drag during the electrophoresis and
detecting the mRNA, based on separation properties of the tagged mRNA.

Embodiment 35: The method of embodiment 34, wherein the first tag and/or the second tag are attached to the sample mRNA using an annealing procedure.

Embodiment 36: The method of embodiment 34, wherein the annealing procedure comprises incubating the sample mRNA with the first tag and/or the second tag under hybridization conditions.

Embodiment 37: The method of embodiment 36, wherein the hybridization conditions comprise a temperature of 73-77° C., about 25 mM KCl in a buffer.

Embodiment 38: The method of embodiment 34, wherein the tagged mRNA is added to a buffer solution.

Embodiment 39: The method of embodiment 38, wherein the tagged mRNA is added to the buffer solution by an injection method.

Embodiment 40: The method of embodiment 38, wherein the injection method is a pressure injection.

Embodiment 41: The method of embodiment 38, wherein the injection method is an electrokinetic injection.

Embodiment 42: The method of embodiment 37, wherein the buffer solution comprises a plurality of reagents comprising a first surfactant and a second surfactant.

Embodiment 43: The method of embodiment 37, wherein the buffer solution is a CiEJ buffer solution optionally containing urea.

Embodiment 44: The method of embodiment 43, wherein the first surfactant is pentaethylene glycol monododecyl ether.

Embodiment 45: The method of embodiment 43, wherein the second surfactant is pentaethylene glycol monodecyl ether.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for detecting a mRNA in a mixture, the method comprising:
subjecting a liquid sample comprising the mRNA to mobility or charge based separation, wherein the mRNA comprises a first tag that increases hydrodynamic drag during the mobility or charge based separation and
detecting the mRNA.

2. The method of claim 1, wherein the first tag comprises a first nucleic acid sequence that is complementary to a first polynucleotide segment of the mRNA.

3. The method of claim 2, wherein the first polynucleotide segment comprises at least a first portion of a first untranslated region (UTR) of the mRNA.

4. The method of claim 3, wherein the first UTR is a 5′UTR.

5. The method of claim 3, wherein the first UTR is a 3′ UTR.

6. The method of any one of claims 2-5, wherein the first tag comprises a first hydrophobic region linked to the first nucleic acid sequence.

7. The method of claim 6, wherein the first hydrophobic region comprises an alkyl group.

8. The method of claim 7, wherein the alkyl group is linear or branched.

9. The method of claim 7 or 8, wherein the alkyl group is saturated.

10. The method of any one of claims 7-9, wherein the alkyl group comprises 8-24 carbon atoms, 12-24 carbon atoms, 15-24 carbon atoms, 18-24 carbon atoms, 8-18 carbon atoms, 12-18 carbon atoms, 15-18 carbon atoms, 8-10 carbon atoms, or 8-12 carbon atoms.

11. The method of any one of claims 6-10, wherein the first hydrophobic region comprises a C18 molecule.

12. The method of any one of claims 6-10, wherein the first hydrophobic region comprises two C18 molecules.

13. The method of any one of claims 2-12, wherein the first nucleic acid sequence comprises a DNA oligonucleotide, optionally comprising a DNA base modification, LNA base modification, or 2′ Ome base modification.

14. The method of any one of claims 1-13, wherein the mRNA comprises a second tag.

15. The method of claim 14, wherein the second tag comprises a second nucleic acid sequence that is complementary to a second polynucleotide segment of the mRNA.

16. The method of claim 14 or 15, wherein the second polynucleotide segment comprises at least a second portion of a second untranslated region (UTR) of the mRNA comprises, wherein optionally the first UTR and second UTR are the same or different UTRs.

17. The method of claim 16, wherein the second UTR is a 5′UTR.

18. The method of claim 16, wherein the second UTR is a 3′ UTR.

19. The method of any one of claims 15-18, wherein the second tag comprises a detectable tag attached to the second nucleic acid sequence, optionally wherein the detectable tag is a non-fluorescent tag.

20. The method of claim 19, wherein the detectable tag is a fluorescent tag, and wherein the fluorescent tag is optionally selected from a 6-Carboxyfluorescein (6-FAM), 5-TAMRA, Cy3, Cy5, Fluorescein, TYE 563, TYE 664, TYE 705, or Yakima Yellow fluorescent tag.

21. The method of claim 14, wherein second tag comprises an RNA dye.

22. The method of claim 15, wherein the second tag comprises a second hydrophobic region linked to the second nucleic acid sequence.

23. The method of claim 22, wherein the second hydrophobic region comprises an alkyl group.

24. The method of any one of claims 1-23, wherein the mobility or charge based separation is a free solution capillary electrophoresis assay.

25. The method of claim 24, wherein the free solution capillary electrophoresis assay is an end-labeled free solution electrophoresis (ELFSE) assay.

26. The method of claim 24, wherein the free solution capillary electrophoresis assay is a micellar end-labeled free solution electrophoresis (miELFSE) assay.

27. The method of any one of claims 1-26, wherein the mRNA is a full-length mRNA.

28. The method of claim 27, wherein the full-length mRNA is tagged with the first tag and the second tag.

29. The method of claim 28, wherein the first polynucleotide segment comprises at least a portion of a 5′UTR of the mRNA and the second polynucleotide segment comprises at least a portion of a 3′UTR of the mRNA.

30. The method of claim 28, wherein the first polynucleotide segment comprises at least a portion of a 3′UTR of the mRNA and the second polynucleotide segment comprises at least a portion of a 5′UTR of the mRNA.

31. The method of any one of claims 1-30, wherein detecting the mRNA comprises detecting separation properties of the mRNA based on mobility and detecting a spectral signal.

32. The method of any one of claims 1-31, wherein the liquid sample comprises 1-20 mRNAs, and wherein the first tag on at least one of the mRNAs is distinct from the first tag on at least one of the other mRNAs.

33. The method of any one of claims 1-32, wherein the first tag and/or the second tag are attached to the sample mRNA using an annealing procedure.

34. The method of claim 33, wherein the annealing procedure comprises incubating the sample mRNA with the first tag and/or the second tag under hybridization conditions.

35. The method of claim 34, wherein the hybridization conditions comprise a temperature of 73-77° C., about 25 mM KCl in a buffer.

36. The method of any one of claims 1-32, wherein the tagged mRNA is added to a buffer solution.

37. The method of claim 36, wherein the tagged mRNA is added to the buffer solution by an injection method.

38. The method of claim 37, wherein the injection method is a pressure injection.

39. The method of claim 37, wherein the injection method is an electrokinetic injection.

40. The method of claim 36, wherein the buffer solution is a CiEJ buffer solution, optionally containing urea.

41. The method of claim 36, wherein the buffer solution comprises a plurality of reagents comprising a first surfactant and a second surfactant.

42. The method of claim 41, wherein the first surfactant is pentaethylene glycol monododecyl ether.

43. The method of claim 41, wherein the second surfactant is pentaethylene glycol monodecyl ether.

44. The method of any one of claims 36-43, wherein the buffer solution interacts with the first tag and/or the second tag, optionally the hydrophobic region of the first tag and/or the second tag to form a drag tag.

45. The method of claim 44, wherein the drag tag is a micelle drag tag, a protein drag tag, a polymeric nanoparticle drag tag or a metal nanoparticle drag tag.

46. The method of claim 44, wherein the drag tag is selected from a circular triton micelle, a worm-like micelle, a spherical micelle, or a lamellar micelle.

47. The method of claim 44, wherein the drag tag is a cylindrical worm-like micelle.

48. The method of any one of claims 1-47, wherein the mRNA is greater than 500 nucleotides.

49. The method of any one of claims 1-47, wherein the mRNA is 500-15,000 nucleotides, 500-12,000 nucleotides, 500-10,000 nucleotides, 500-8,000 nucleotides, 1,000-15,000 nucleotides, 1,000-12,000 nucleotides, 1,000-10,000 nucleotides or 1,000-8,000 nucleotides.

50. A composition comprising a nucleic acid sequence that is complementary to a polynucleotide segment of a mRNA and a hydrophobic region linked to the nucleic acid sequence, wherein the hydrophobic region comprises an alkyl group.

51. The composition of claim 50, wherein the polynucleotide segment comprises at least a portion of an untranslated region (UTR) of the mRNA.

52. The composition of claim 51, wherein the UTR is a 5′UTR.

53. The composition of claim 51, wherein the UTR is a 3′ UTR.

54. The composition of any one of claims 50-53, wherein the alkyl group is linear.

55. The composition of any one of claims 50-54, wherein the alkyl group is saturated.

56. The composition of any one of claims 50-55, wherein the alkyl group comprises 8-24 carbon atoms, 12-24 carbon atoms, 15-24 carbon atoms, 18-24 carbon atoms, 8-18 carbon atoms, 12-18 carbon atoms, 15-18 carbon atoms, 8-10 carbon atoms, or 8-12 carbon atoms.

57. The composition of any one of claims 50-53, and 55-56, wherein the alkyl group is branched.

58. The composition of any one of claims 50-57, wherein the hydrophobic region comprises a C18 molecule, two C18 molecules, or more than two C18 molecules.

59. The composition of any one of claims 50-58, wherein the hydrophobic region linked to the nucleic acid sequence comprises a DNA base modification, LNA base modification, or 2′ Ome base modification.

60. A construct comprising:
(i) a mRNA polynucleotide,
(ii) a first tag comprising a hydrophobic region linked to a first nucleic acid sequence hybridized to the mRNA polynucleotide, wherein the first nucleic acid sequence is complementary to a first polynucleotide segment of the mRNA and
(iii) a second tag comprising detectable molecule linked to a second nucleic acid sequence hybridized to the mRNA polynucleotide, wherein the second nucleic acid sequence is complementary to a second polynucleotide segment of the mRNA.

61. A method for detecting a mRNA, the method comprising:
(a) attaching a first tag and a second tag to a mRNA molecule to generate a tagged mRNA molecule,
(b) subjecting the tagged mRNA molecule to a capillary electrophoresis assay, wherein the first tag causes a change in separation properties of the mRNA molecule in the assay to separate the mRNA molecule from other components of the mixture, and
(c) detecting a signal corresponding to the second tag based on the separated mRNA molecule, and thereby identifying a signal corresponding to the mRNA.
